(19) United States
(12) Patent Application Publication
(10) Pub. No.: US 2010/0280563 A1
(43) Pub. Date: Nov. 4, 2010

(54) DEVICE AND METHOD FOR DETECTING AND TREATING A MYOCARDIAL INFARCTION USING PHOTOBIOMODULATION

(76) Inventors: Anne Norlin-Weissenrieder, Stockholm (SE); Leda Henriquez, Bandhagen (SE); Hans Strandberg, Sundbyberg (SE); Eva Hartström, Hasselby (SE); Mikael Sjögren, Fjardhundra (SE); Annika Naeslund, Bromma (SE); Johan Eckerdal, Knivsta (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/528,363

(22) PCT Filed: Feb. 28, 2007
(86) PCT No.: PCT/SE07/00194

§ 371 Date: May 20, 2010

(51) Int. Cl. A61N 1/362

(52) U.S. Cl. 607/3

(57) ABSTRACT

In an implantable medical device and a method for treating cardiac tissue of a heart of a patient with therapeutic light, a myocardial infarction is detected and a location the myocardial infarction is identified. A therapy session is initiated by selectively activating one or more of a number of light emitting units arranged in at least one medical lead connectable to the implantable medical device, to emit therapeutic light toward the detected location of the myocardial infarction upon detection of an occurrence of the myocardial infarction.

### BACKGROUND OF THE INVENTION

1. Field of the Invention

The present invention generally relates to cardiac pacing systems and, in particular, to methods and medical devices for detecting and treating myocardial infarctions.

2. Description of the Prior Art

Due to the in general poorer medical status of pacemaker and ICD patients they are subjected to an increased risk of myocardial infarction (MI). The term myocardial infarction refers to the death of myocardial or heart tissue caused by a partial or complete blockage of in one the arteries that supply blood to the heart (coronary arteries), resulting in an interruption in the blood supply to the heart. In the classical acute MI there is a sudden occlusion of a coronary artery due to thrombosis resulting in the death of part of either the right or left ventricular wall. The thrombus occurs due to atheromatous changes in the blood vessel wall.

When heart tissue is deprived of blood-borne oxygen for longer than 30 minutes (called ischemia), it begins to die. Ischemia causes electrical instability within the chambers of the heart, preventing the heart from adequately pump blood throughout the body.

Cardiac repair after MI is a complex process involving diverse inflammatory components, extracellular matrix remodelling and responses of the cardiomyocytes to ischemia. After necrosis of the cardiomyocytes and a long inflammatory phase, the ischemic zone is subsequently replaced by fibrotic tissue. This permanent damage of the heart muscle increases the risk of developing congestive heart failure (CHF).

It is critical to begin treatment of the areas affected by ischemia as soon as possible after the myocardial infarction. Intensive research over the last 20 or more years has demonstrated that prompt treatment can decrease damage from a heart attack and increase the chance for survival. If such therapy is initiated within 1 hour of the inset of symptoms, less irreparable damage may occur.

In light of this, a number of approaches have been made to detect and/or to treat myocardial infarction in implantable medical devices such as pacing devices. For example, in EP 467 695 A2 a method and apparatus for detecting and treating myocardial infarctions in antitachy-arrhythmia and bradycardia pacing devices are disclosed. Electrical activity of the patient's heart is sensed and signalled in order to detect the presence of an MI and a thrombolytic drug is released into the bloodstream upon such detection. Thus, this solution improves the supply of blood at the detection of an MI but, however, it does not treat potential damages of the cardiac tissue caused by the MI.

In EP 1 384 433, by the same applicant, a monitor for early detection of an ischemic heart disease of a patient using intracardiac impedance is shown. According to this solution, the impedance changes due to the increased stiffness of the cardiac tissue caused by the ischemic heart disease are detected. However, EP 1 384 433 is not concerned with the treatment of a detected ischemia.

Furthermore, EP 1 690 566, U.S. Pat. No. 6,604,000 and U.S. Pat. No. 6,256,538 also present implantable medical devices incorporating an ischemia detector responsive to measured intracardiac impedance.

US 2004/0260367 shows a method for treating a detected myocardial infarction of a patient's heart. According to this solution, a light source adapted to generate therapeutic light in the visible to near-infra-red wavelength range using so called low level light therapy (“LLLT”) or phototherapy is positioned relative to the patient's heart on the torso of the patient. The therapeutic light penetrates the intervening tissue and the cardiac tissue is irradiated according to a treatment protocol. Thus, the solution according to US 2004/0260367 is impaired with the problem that a detection of the myocardial infarction and a determination of the location of the myocardial infarction are required before the treatment can be initiated. As discussed above, the heart tissue begins to die if it is deprived of blood-borne oxygen for longer than 30 minutes and hence it is critical to begin treatment of the areas affected by ischemia as soon as possible after the myocardial infarction. Therefore, the cardiac tissue may already have been affected with damages, which may be irreparable, when the treatment can be initiated.

Thus, there remains a need within the art of a method and medical device that are capable of detecting the occurrence and location of a myocardial infarction and initiating a treatment of the cardiac tissue suffering from the myocardial infarction subsequently to the detection.

### SUMMARY OF THE INVENTION

An object of the present invention is to detect the occurrence and location of a myocardial infarction is detected and to administer a treatment to the cardiac tissue suffering from the myocardial infarction is initiated.

According to another object of the present invention, the occurrence and location of a myocardial infarction is automatically detected and a treatment of the cardiac tissue suffering from the myocardial infarction is automatically initiated subsequently to the detection.

According to a further object of the present invention, a commencement of a myocardial infarction and the location of the myocardial infarction can be detected at an early stage.

According to an aspect of the present invention, there is provided an implantable medical device including a pulse generator emits cardiac stimulating pacing pulses and that is connectable to at least one medical lead for delivering the pulses to cardiac tissue of a heart of a patient. The implantable medical device has a myocardial infarction detection means, which myocardial infarction detection unit that detects a myocardial infarction and identifies a location of the myocardial infarction. Further, the implantable medical device has therapy circuitry connected to a number of light emitting units arranged in the at least one medical lead adapted to emit therapeutic light, and a control circuit connected to the myocardial infarction detection unit and to the light emitting units, the control circuit being configured to initiate a therapy session in which one or more of the light emitting units is/are selectively activated to emit the therapeutic light toward a detected location of the myocardial infarction upon detection of an occurrence of the myocardial infarction.

According to a second aspect of the present invention, there is provided a method for treating cardiac tissue of a heart of a patient with therapeutic light using an implantable medical device including a pulse generator adapted to produce cardiac stimulating pacing pulses and being connectable to at least one medical lead for delivering the pulses to cardiac tissue of a heart of a patient. The method includes the steps of intracorporeally detecting a myocardial infarction and identifying a location of the myocardial infarction, and initiating a therapy session by selectively activating one or more of a number of intracorporeally placed light emitting units arranged in the at least one medical lead to emit therapeutic light toward the detected location of the myocardial infarction upon detection of an occurrence of the myocardial infarction.

According to a third aspect of the present invention, there is provided a computer-readable medium, directly loadable into an internal memory of an implantable medical device according to the first aspect of the present invention, encoded with software code that causes the implantable medical device to perform steps in accordance with a method according to the second aspect of the present invention.

The invention utilizes the technique photobiomodulation, also called Low Level Laser Therapy (LLLT), Cold Laser Therapy (CLT), Laser Biomodulation, phototherapy or Laser therapy, wherein certain wavelengths of light at certain intensities are delivered for a certain amount of time. More specifically, the present invention is based on the insight of using such therapeutic light to treat cardiac tissue after a myocardial infarction. This is based upon the findings that photobiomodulation has been proven to be a successful therapy in wound healing see, for example, “Effect of NASA light-emitting diode irradiation on wound healing”, H. T. Whelan et al., Journal of Clinical Laser Medicine and Surgery, 19, (2001) p 305. It was also confirmed by Whelan et al. that the cell growth of various cell types in human and rat could be increased by up to 200% by irradiation of light of certain wavelengths. Furthermore, it has also been shown, for example, in “Low energy laser irradiation reduces formation of scar tissue after myocardial infarction in rats and dogs”, U. Oron, et al., Circulation, 103, (2001), p 296, that light therapy improves the regeneration of the cardiac cells and decreases the scar tissue formation following a myocardial infarction.

Thus, the present invention provides a number of advantages, for example, an occurrence and location of a myocardial infarction can be detected at an early stage and the treatment of the myocardial infarction can thus be initiated at an early stage. This is of high importance since it has been shown that it is critical to initiate the treatment of the areas of cardiac tissue affected by ischemia as soon as possible after the myocardial infarction. Intensive research over the last 20 or more years has demonstrated that prompt treatment may decrease damage from a heart attack and increase the chance for survival. If a therapy is initiated within 1 hour of the onset of the infarct, less irreparable damage may occur. A further advantage of the present invention is that the regeneration of cardiac cells after a myocardial infarction is improved.

According to an embodiment, the therapy circuit is adapted to activate the light emitting unit or units to emit the therapeutic light according to a treatment protocol, wherein the treatment protocol includes treatment parameters comprising: emitting intervals of the therapeutic light, intensity of the emitted therapeutic light, wavelength of the emitted light, and/or intermittence of the emitted therapeutic light.

In one embodiment, each of the light emitting units is formed by at least one light emitting diode. The light emitting units, according to other embodiments, may be arranged in an array along an outer surface of a lead body of respective medical lead.

According to an embodiment of the present invention, the electrodes are arranged in an array along the outer surface of a lead body of respective medical lead.

In a further embodiment, each of the light emitting units includes at least one optical fiber adapted to conduct light emitted from at least one light source arranged in the implantable medical device, and the therapy circuit selectively activates the at least one light source and/or at least one optical fibre such that light conducted in one or more optical fibres emanates from the one or more optical fibers toward the detected location.

The at least one light source may be a laser source adapted to emit coherent and monochromatic light having a wavelength in the range of 600 nm-1000 nm. Furthermore, in one embodiment, an intensity of 1-500 mW/cm² and a total dosage of about 1-4 J/cm² are applied. In another embodiment, an intensity of 6-50 mW/cm² and a total dosage of about 1-4 J/cm² may be applied.

According to an embodiment of the present invention, the myocardial infarction detection unit includes an impedance measuring circuit connected to the electrodes arranged in the medical leads. The impedance measuring device is adapted to apply excitation current pulses between respective electrode pairs including at least a first and at least a second electrode and to measure the impedance in the tissues between the at least first and the at least second electrode of the electrode pairs to the excitation current pulses. Further, the myocardial infarction detection means includes a myocardial infarct detector adapted to evaluate the measured impedances by detecting changes in the impedances being consistent with a myocardial infarction and to determine a location of the myocardial infarction using the evaluation. The impedance measuring circuit may measure the impedance between a number of different combinations of electrodes. The impedance measuring circuit may be adapted to periodically initiate impedance measuring sessions according to a myocardial infarction monitoring protocol, wherein the impedance between different pairs of electrodes is measured according to a predetermined sequence (e.g. one pair after another during consecutive cardiac cycles or all pairs simultaneously during a number of consecutive cardiac cycles) to be able to detect and locate a myocardial infarction. That is, during each impedance measuring session, a number of impedance measurements from the different electrode pairs are obtained. Consequently, it is possible to continuously monitor the cardiac tissue to enable a reliable detection of the occurrence and location of a myocardial infarction.

According to embodiments of the present invention, the myocardial infarct detector compares measured impedances with a stored reference impedance template to detect an occurrence of a myocardial infarction and a location of the myocardial infarction from the result of the comparison. The template may alternatively be obtained or created by the myocardial infarction detection means during a period when no changes of the monitored signals, e.g. impedance or electrical activity of the cardiac tissue, are of a sufficient magnitude to indicate the possibility of the commencement of a condition such as a myocardial infarction. Such a template may also be updated periodically by performing new measurements of the impedance and/or the electrical activity.

In one example, impedance value ratios for a cardiac cycle is determined by determining a maximum impedance and a minimum impedance, respectively, measured by the impedance measuring circuit during a cardiac cycle. Further, an impedance value ratio being below a predetermined impedance value ratio threshold is determined to be consistent with a myocardial infarction; and the impedance value ratio being smallest of the impedance value ratios being below the predetermined impedance value ratio threshold is determined to indicate the location of the myocardial infarction.

Alternatively, or as a complement to the impedance value ratio determination, the myocardial infarct detector may be adapted to calculate a respective maximum time derivative of the measured impedance curves, to determine a maximum impedance time derivative being below a predetermined impedance time derivative threshold to be consistent with a myocardial infarction and to determine the maximum impedance time derivative being lowest of the maximum impedance time derivatives being below the predetermined impedance time derivative threshold to indicate the location of the myocardial infarction.

In yet another embodiment of the present invention, the myocardial infarction detection unit has an intracardiac electrogram measuring circuit connected to the electrodes of respective medical leads and which measuring circuit is adapted to measure intracardiac electrograms using one or more electrodes of the medical leads. Furthermore, the myocardial infarction detection unit includes a myocardial infarct detector adapted to evaluate the intracardiac electrograms to detect changes being consistent with a myocardial infarction and to determine a location of the myocardial infarction using the evaluation. A reference template, which template may be a stored reference impedance template, may be used in this evaluation. The template may alternatively be obtained or created by the myocardial infarction detection means during a period when no changes of the monitored signals, e.g. the electrical activity of the cardiac tissue, are of a sufficient magnitude to indicate the possibility of the commencement of a condition such as a myocardial infarction. Such a template may also be updated periodically by performing new measurements of the electrical activity. Consequently, it is possible to continuously monitor the cardiac tissue to enable a reliable detection of an occurrence and location of a myocardial infarction.

In a specific embodiment of the present invention, the myocardial infarct detector is adapted to determine a ST segment elevation being above a predetermined ST segment threshold as being consistent with the occurrence of a myocardial infarction and determine the intracardiac electrogram having the largest ST segment elevation of the ST segments being above a predetermined ST segment threshold as indicating the location of the myocardial infarction.

Furthermore, according to embodiments of the present invention, a combination of impedance measurements and intracardiac electrograms is used to detect an occurrence and location of a myocardial infarction. For example, both ST segment elevations and maximum impedance time derivatives may be used to detect myocardial infarctions. Thereby, it is possible to obtain a more reliable detection of the myocardial infarction and the location of the myocardial infarction.

At an infarction, certain hormones or chemical substances are released or are produced in a higher concentration than normal, for example, creatine phosphatinase, FABP (Fatty Acid Bonding Proteins), LDH (Lactic Dehydrogenase), or GOT (Glutamic-Oxalatic Transaminase). In one embodiment of the present invention, this is utilized by arranging a sensor in the implantable medical device or in the medical leads adapted to sense such a hormone or substance. A semiconductor sensor may be used where a reactance material is applied on a surface of the sensor, which reactance material is specific to react with the substance of interest.

According to embodiments of the present invention, signals being indicative of the healing process of the myocardial infarction is monitored, continuously or periodically, during the therapy session to determine whether the therapy has been successful and should be ended or whether the therapy parameters, i.e. the parameter of the treatment protocol, should be adjusted in order to make the treatment more potent during a certain phase of the healing process or to make the treatment less potent. A more potent treatment may be a higher degree of intensity of light or a constant intensity of light but with a changed intermittence, i.e. longer periods of light delivery or a more frequent light delivery with a constant period of light delivery. A less potent treatment may instead be a lower degree of intensity of light or a constant intensity of light but with a changed intermittence, i.e. shorter periods of light delivery or a less frequent light delivery with a constant period of light delivery.

According to one embodiment of the present invention, the myocardial infarct detector, after an initiation of a therapy session, monitors impedances obtained by at least an electrode pair indicating the location of the myocardial infarction to determine whether the impedances indicate that the therapy session should be terminated and/or the treatment parameters should be adjusted or maintained.

Furthermore, the myocardial infarct detector may be adapted to determine impedance value ratios for successive cardiac cycles and to determine that the therapy session should be terminated if a predetermined number of the impedance value ratios are found to be above the impedance value ratio threshold.

In another embodiment, the myocardial infarct detector may be adapted to calculate maximum time derivatives of the measured impedance curves for successive cardiac cycles and to determine that the therapy session should be terminated if a predetermined number of the maximum impedance time derivatives are found to be above a predetermined impedance time derivative threshold.

According to further embodiments, the myocardial infarct detector is adapted to monitor intracardiac electrograms obtained by at least an electrode pair indicating the location of the myocardial infarction to determine whether the intracardiac electrograms indicate that the therapy session should be terminated and/or the treatment parameters should be adjusted or maintained.

In a certain embodiment, the myocardial infarct detector is adapted to determine ST segments for successive cardiac cycles and determine that the therapy session should be terminated if a predetermined number of the ST segment elevations are found to be below a predetermined ST segment elevation threshold.

Moreover, according to other embodiments of the present invention, a combination of impedance measurements and intracardiac electrograms is used to determine whether the therapy should be terminated or whether the therapy parameters should be adjusted or maintained. For example, both ST segment elevations and maximum impedance time derivatives may be used to evaluate the therapy. Thereby, it is possible to obtain a more reliable judgement of the healing process and the therapy.

According to an embodiment of the present invention, the implantable medical device is provided with a power transmission unit that operates by inductive coupling in order to provide the implantable medical device with additional energy for a healing process. A receiver coil with a rectifier is arranged in the implantable medical device. An external sending coil is arranged to emit AC-fields in frequencies of a few kHz to about 500 kHz. This additional energy may be supplied directly to the light emitting means, for example, the diodes or may be used to charge a re-chargeable battery of the implantable medical device.

In a further embodiment, a warning system is arranged in the implantable medical device adapted to notify the patient (e.g. by means of a beep signal or a generated vibration) and/or a care institution such a hospital. For example, the hospital can be notified via message transmitted via an RF (Radio Frequency) unit of the implantable medical device and telecommunication system containing, inter alia, information related to the patient and a detected myocardial infarction stored in the implantable medical device. A decision at the hospital how to proceed with the treatment of the infarct can be based on the transmitted information collected by the sensors of the implantable medical device. For example, medical personnel is able to tune the light therapy by programming the device and the device can be provided with additional power or energy can be supplied from an external power source shortly after the onset of the infarct. The patient is also able to contact medical personnel via a home monitoring equipment installed at his/hers home at notification of a detection of an infarct.

In one embodiment of the present invention, the light emitting units are activated such that therapeutic light is emitted according to a treatment protocol including treatment parameters comprising one, more or all of: emitting intervals of the therapeutic light, intensity of the emitted therapeutic light, wavelength of the emitted light, intermittence of the emitted therapeutic light, or treatment periods. The protocol may thus comprise a predetermined treatment scheme. In an alternative embodiment, the treatment is varied in dependence of one or more treatment response parameters.

In embodiments of the present invention, the light emitting units emit coherent and monochromatic light having a wavelength in the range of 600 nm-1000 nm. Furthermore, an intensity of 1-500 mW/cm² and a total dosage of about 1-4 J/cm² may be used.

As will be apparent to those skilled in the art, steps of the method of the present invention, as well as preferred embodiment thereof, are suitable to realize as a computer program or an encoded computer readable medium.

### BRIEF DESCRIPTION OF THE DRAWINGS

The features that characterize the invention, both as to organization and to method of operation, together with further objects and advantages thereof, will be better understood from the following description used in conjunction with the accompanying drawings. It is to be expressly understood that the drawings is for the purpose of illustration and description and is not intended as a definition of the limits of the invention. These and other objects attained, and advantages offered, by the present invention will become more fully apparent as the description that follows is read in conjunction with the accompanying drawings.

FIG. 1 schematically shows an embodiment of a pacemaker system in which an implantable medical device in accordance with the present invention may be implemented.

FIG. 2a schematically illustrates an embodiment of the implantable medical device according to the present invention.

FIG. 2b schematically illustrates another embodiment of the implantable medical device according to the present invention.

FIG. 3a schematically illustrates an embodiment of the myocardial infarction detection unit in accordance with the present invention.

FIG. 3b schematically illustrates another embodiment of the myocardial infarction detection unit in accordance with the present invention.

FIG. 4 schematically illustrates an embodiment of a medical lead in accordance with the present invention.

FIG. 5 is high-level flow chart of an embodiment of the method for treating a myocardial infarction with therapeutic light using an implantable medical device according to the present invention.

In the following, the present invention will be discussed in the context of medical systems including at least an implantable pacemaker, and medical leads such as an atrial lead and a ventricular lead.

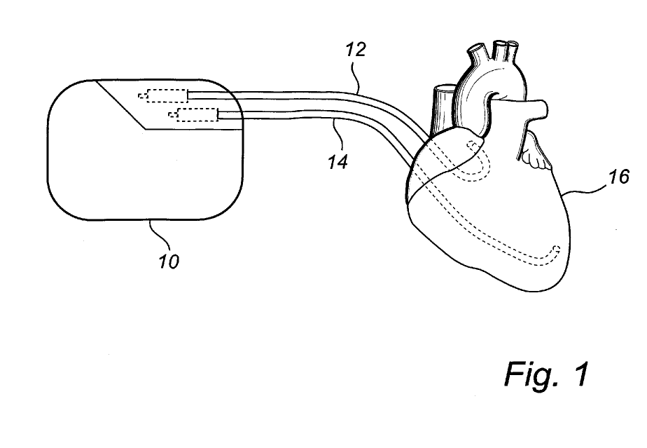

With reference first to FIG. 1, a pacemaker system is shown that includes an implantable pacemaker 10 connectable to an atrial lead 12 and a ventricular lead 14 including electrodes for providing therapy to a heart 16 of a patient. The leads 12, 14 are implanted into the heart 16 via veins and are fixated at the cardiac tissue by means of, for example, helical screws.

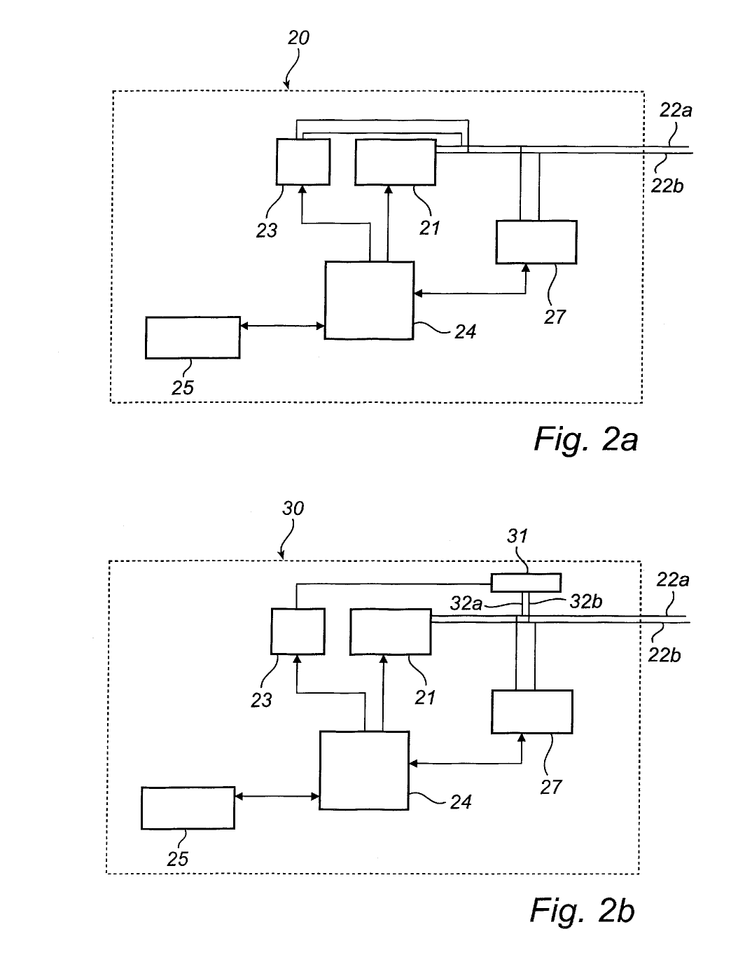

Turning now to FIGS. 2a, an embodiment of an implantable medical device, e.g. a pacemaker or an ICD, according to the present invention will be discussed. The implantable medical device 20 comprises a housing (not shown) being hermetically sealed and biologically inert. Normally, the housing is conductive and may, thus, serve as an electrode. The pacemaker 20 is connectable to one or more pacemaker leads, where only two are shown in FIGS. 2a and 2b; namely a ventricular lead 22a implanted in the right ventricle of the heart (not shown) and one atrial lead 22b implanted in the right atrium of the heart (not shown).

The leads 22a and 22b can be electrically coupled to the pacemaker 20 in a conventional manner. The leads 22a, 22b carry one or more electrodes, such as a tip electrode or ring electrodes, arranged to, inter alia, measure the impedance or transmit pacing pulses for causing depolarization of cardiac tissue adjacent to the electrode(-s) generated by a pace pulse generator 21 under influence of a controller or controlling circuit 24 including a microprocessor. The controller 24 controls, inter alia, pace pulse parameters such as output voltage and pulse duration.

Moreover, a storage unit 25 is connected to the controller 24, which storage unit 25 may include a random access memory (RAM) and/or a non-volatile memory such as a read-only memory (ROM). The storage unit 25 is connected to the controller 24. Detected signals from the patient's heart are processed in an input circuit (not shown) and are forwarded to the controller 24 for use in logic timing determination in known manner.

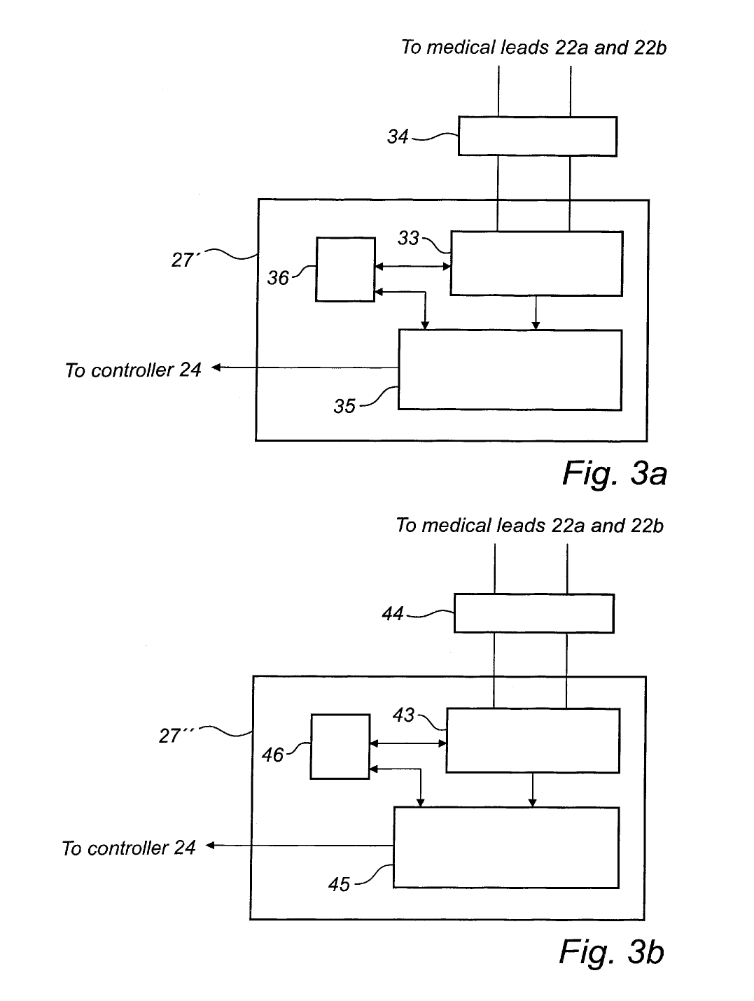

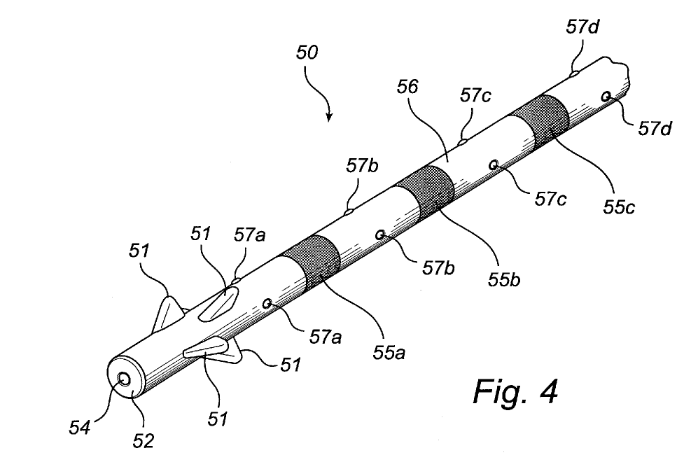

Furthermore, the implantable medical device 20 has a myocardial infarction detection unit 27, which will be described below in more detail with reference to FIGS. 3a and 3b. The myocardial infarction detection unit 27 is configured to process detected signals from the patient's heart to detect whether a myocardial infarction has occurred and may also, if such an infarction is detected, determine or identify a location of the detected myocardial infarction within the heart. Information from the myocardial infarction detection unit 27 such as detection of a myocardial infarction and the location may be forwarded to the controller 24. The myocardial infarction detection unit 27 is connected to the electrodes arranged in the medical leads 22a, 22b, for example, a number of ring electrodes arranged along the medical leads and tip electrodes, see FIG. 4.

In this embodiment, a plurality of light emitting units (see FIG. 4) are incorporated in one or all of the leads 22a, 22b and connected to the controller 24. In one embodiment, the light emitting units are formed of light emitting diodes that are arranged at a periphery of the tube-shaped leads 22a and 22b in an array along a longitudinal direction of the leads. The light emitting diodes emit monochromatic light having a wavelength of 600-1000 nm. The light emitting diodes are connected to a therapy circuit 23, which is adapted to, under control of the controller 27, selectively activate one or more of the diodes. Upon a detection of a myocardial infarction and at determination of a location within the heart of such an infarction, the myocardial infarction detection unit 27 may forward this information to the controller 24, which, in turn, may activate selected diodes via the therapy circuit 23 according to a treatment protocol. One or more of the diodes may be selected, based on the determination of the location of the myocardial infarction, and activated to emit therapeutic light towards the detected myocardial infarction. The treatment protocol may include predetermined or adjustable treatment parameters such as emitting intervals of the therapeutic light, intensity of the emitted therapeutic light, wavelength of the emitted light, and intermittence of the emitted therapeutic light.

The implantable medical device 20 is powered by a battery (not shown), which supplies electrical power to all electrical active components of the implantable medical device 20 including the light emitting units arranged in the medical leads 22a and 22b and the myocardial infarction detection unit 27. The implantable medical device 20 may also be provided with means for power transmission via inductive coupling in order to provide the implantable medical device 20 with additional energy for a healing process. A receiver coil (not show) with a rectifier is arranged in the implantable medical device 20. An external sending coil is arranged to emit AC-fields in frequencies of a few kHz to about 500 kHz. This additional energy may be supplied directly to the light emitting units, for example, the diodes or may be used to charge a re-chargeable battery of the implantable medical device 20.

The implantable medical device 20 further has a communication unit (not shown), for example, an RF telemetry circuitry for providing RF communications. Thereby, for example, data contained in the storage means 25 can be transferred to an external programmer device (not shown) via the communication unit and a programmer interface (not shown) for use in, for example, analyzing system conditions, patient information, etc.

Moreover, the implantable medical device 20 may further has a notifying device (not shown) adapted to, at detection of an occurrence of a myocardial infarction, notify said patient of the event that a myocardial infarct has been detected and/or that therapy has been initiated. In one embodiment, the notifying device is a vibration unit adapted to vibrate in the event that a myocardial infarct has been detected and/or that therapy for treating such an infarct has been initiated and thereby notify the patient.

Referring to FIG. 2b, a further embodiment of the implantable medical device according to the present invention will be discussed. Like parts in the implantable medical device shown in FIG. 2a and FIG. 2b will be denoted with the same reference numerals and descriptions thereof will be omitted since they have been described above with reference to FIG. 2a. A light source 31 is arranged in the implantable medical device 30, for example, a laser adapted to emit monochromatic light having a wavelength of 600-1000 nm. The light source 31 is connected to a number of optical fibers 32a arranged in the ventricular lead 22a and a number of optical fibers 32b arranged in the atrial lead 22b. The optical fibers 32a, 32b are arranged to conduct light emitted by the light source 31 such that the conducted therapeutic light emanates from the optical fibers 32a, 32b toward the cardiac tissue. The optical fibers 32a, 32b are arranged such that light can be applied cardiac tissue along the periphery of the medical leads 22a, 22b. That is, distal ends of respective optical fibers 32a, 32b are arranged in arrays along the outer periphery of the medical leads 22a, 22b see FIG. 4. Furthermore, the light source 31 has a selector circuit adapted to, under influence of the controller 27, select one or more of the optical fibers 32a, 32b to conduct light during a therapy session such that therapeutic light can be applied to an identified location of a myocardial infarction.

Referring now to FIGS. 3a and 3b, embodiments of the myocardial infarction detection means will be discussed in more detail. With reference first to FIG. 3a, an embodiment of the myocardial infarction detection means adapted to determine an occurrence of a myocardial infarction and to determine the location of the myocardial infarction using measured impedances, for example, transcardiac impedances will be described. The myocardial infarction detection unit 27′ has an impedance measuring circuit 33 connected to the electrodes incorporated in the medical leads 22a and/or 22b, which will be described in more detail below with reference to FIG. 4. In one embodiment, each medical lead carries ring electrodes arranged along the respective lead and a tip electrode and the impedance measuring circuit 33 may be connected to the electrodes via a switching device 34. The switching device 34 may be arranged in the implantable medical device 20 and are adapted to switch an applied current to a selected electrode(-s) of the medical lead(-s) 22a, 22b. Those skilled in the art may design such a switching device based on the switching device described in U.S. Pat. No. 5,423,873, the teaching of which hereby are incorporated by reference in its entirety. Hence, the impedance measuring circuit 33 may, for example, measure the impedance between a first ring electrode of the first medical lead 22a and the housing the implantable medical device, a first ring electrode of the first medical lead 22a and a second ring electrode of the first medical lead 22a, a first ring electrode of the first medical lead 22a and a first ring electrode of the second medical lead 22b, and first ring electrode of the second medical lead 22b and a second ring electrode of the second medical lead 22b. The impedance measuring circuit 33 is adapted to apply excitation current pulses between respective electrode pair including at least a first and at least a second electrode, as mentioned above, to measure the impedance in the tissues between the at least first and the at least second electrode of the respective electrode pairs to the excitation current pulses. The impedance measuring circuit 33 is adapted to periodically initiate impedance measuring sessions according to a myocardial infarction monitoring protocol, wherein the impedance between different pairs of electrodes is measured according to a predetermined sequence (e.g. one pair after another during consecutive cardiac cycles or all pairs simultaneously during a number of consecutive cardiac cycles) to be able to detect and locate a myocardial infarction. That is, during each impedance measuring session, a number of impedance measurements from the different electrode pairs are obtained. For example, a measurement including four electrode pairs will provide four impedance values.

Furthermore, myocardial infarction detection means 27′ comprises a myocardial infarct detector 35 adapted to evaluate the measured impedances by detecting changes in the impedances that is consistent with a myocardial infarction and to determine a location of the myocardial infarction using the evaluation. In one embodiment, the myocardial infarct detector 35 is adapted to compare the measured impedances with a reference impedance template stored in a template memory 36 to detect an occurrence of a myocardial infarction and a location of the myocardial infarction from the result of the comparison. Alternatively, the reference impedance template may be stored in the storage means 25. Moreover, the reference template can be obtained and created before the parameter monitoring session is initiated, e.g. the impedance measurement session, and updated periodically. In one embodiment, the impedance measurements sessions are synchronized with the heartbeats of the patients, for example, at the end of diastole.

The myocardial infarct detector 35 may be adapted to determine impedance value ratios for each cardiac cycle by determining a maximum impedance and a minimum impedance for each electrode pair during the cardiac cycle. By comparison with the template, it is possible to identify whether a myocardial infarction has occurred. For example, an impedance value ratio being below an impedance value ratio threshold is determined to be consistent with a myocardial infarction. Further, by comparing the impedance value ratios being below the threshold, a location of the myocardial infarction can be determined. In this embodiment, the impedance value ratio being smallest is determined to indicate the location of the myocardial infarction. That is, the electrode pair providing the impedance measurement curve having the smallest difference between the maximum impedance value and the minimum impedance value during a cardiac cycle is determined to be the electrode pair being closest to the detected myocardial infarction and, hence, the location of the myocardial can be determined. The myocardial infarct detector 35 is adapted to send an instruction or message to the controller 24 informing the controller 24 that a myocardial infarction has been detected and the location of the myocardial infarction, i.e. as defined by the electrode pair being determined to be closest to the detected myocardial infarction.

In another embodiment of the present invention, the myocardial infarct detector 35 is adapted to calculate a maximum time derivative of each measured impedance curve, i.e. for each electrode pair. By comparing the calculated maximum time derivates with the template, it is possible to identify whether a myocardial infarction has occurred. For example, a maximum impedance time derivative being below a predetermined impedance time derivative threshold is determined to be consistent with a myocardial infarction. Further, in this embodiment, the maximum impedance time derivative being the lowest of the maximum impedance time derivatives being below the impedance time derivative threshold is determined to indicate the location of the myocardial infarction. That is, the electrode pair providing the impedance measurement curve having the lowest maximum impedance time derivative is determined to be the electrode pair being closest to the detected myocardial infarction. The myocardial infarct detector 35 is adapted to send an instruction or message to the controller 24 informing the controller 24 that a myocardial infarction has been detected and the location of the myocardial infarction, i.e. as defined by the electrode pair being determined to be closest to the detected myocardial infarction.

Those skilled within the art appreciate that there are a number of other conceivable variations or alternatives to the embodiments described above.

For example, the morphology of the obtained impedance curves may be compared with an impedance template to determine the occurrence and location of a myocardial infarction. In one embodiment, the part of the impedance curve at systole, i.e. after the QRS-complex, is studied and compared with a reference curve obtained with the same electrode configuration at normal conditions, i.e. at conditions when no myocardial infarction is present.

Moreover, the myocardial infarct detector may be adapted to, after an initiation of a therapy session, monitor impedances obtained by at least the electrode pair that indicated the location of the myocardial infarction to determine whether the obtained impedances indicate that the therapy session should be terminated and/or whether treatment parameters should be adjusted. The therapy parameters can be adjusted during the treatment procedure. For example, a higher light intensity can be used during an initial therapy period and the light intensity can be reduced during a second period after the initial period. Alternatively, a constant light intensity but an adjusted intermittence can be utilized, e.g. the periods of light delivery can be adjusted or shorter intervals between the periods of light delivery are used.

In one embodiment, the myocardial infarct detector is adapted to determine impedance value ratios for successive cardiac cycles and to determine that the therapy session should be terminated if a predetermined number of the impedance value ratios are found to be above a predetermined impedance value ratio threshold. Alternatively, the therapy parameters can be adjusted, for example, shorter intervals between the periods of light delivery can be used if a predetermined number of the impedance value ratios are found to be below a predetermined impedance value ratio threshold.

In a further embodiment, the myocardial infarct detector is adapted to calculate maximum time derivatives of the measured impedance curves for successive cardiac cycles and to determine that the therapy session should be terminated if a predetermined number of the maximum impedance time derivatives are found to be above a predetermined impedance time derivative threshold. Alternatively, the therapy parameters can be adjusted, for example, shorter intervals between the periods of light delivery can be used if a predetermined number of the impedance value ratios are found to be below a predetermined impedance value ratio threshold.

Turning now to FIG. 3b, an embodiment of the myocardial infarction detection unit 27″ adapted to determine an occurrence of a myocardial infarction and to determine the location of the myocardial infarction using electrical activity of the heart of the patient impedances will be described. The myocardial infarction detection unit 27″ 27″ includes a sensor 43 that senses electrical activity of the heart including an intracardiac electrogram measuring circuit connected to electrodes incorporated in the medical leads 22a and/or 22b, which will be described in more detail below with reference to FIG. 4. In one embodiment, each medical lead has a number of ring electrodes arranged along the respective lead and a tip electrode and the means for sensing electrical activity 43 may be connected to the electrodes via a switching device 44. The switching device 44 may be arranged in the implantable medical device 20 and is adapted to switch between selected electrode(-s) of the medical lead(-s) 22a, 22b to obtain signals indicative of the electrical activity of the heart from different electrode(-s) and/or combination of electrodes. Those skilled within the art may design such a switching device based on the switching device described in U.S. Pat. No. 5,423,873, the teachings of which are incorporated herein by reference. Thereby, the electrical activity of the heart can be measured using different electrodes and/or combination of electrodes, for example, at a first ring electrode of the first medical lead 22a and a second ring electrode of the first medical lead 22a, at a first ring electrode of the first medical lead 22a and at a first ring electrode of the second medical lead 22b, and/or at a first ring electrode of the second medical lead 22b and at a second ring electrode of the second medical lead 22b. The electrical activity sensor 43 is adapted to perform sensing sessions according to a myocardial infarction monitoring protocol, wherein the electrical activity at different sensors, electrodes and/or combinations of electrodes are measured according to a predetermined sequence (e.g. one electrode after another during consecutive cardiac cycles or a number of electrodes simultaneously during a number of consecutive cardiac cycles) to be able to sense electrical activity and obtain intracardiac electrograms for different electrodes and/or combinations of electrodes.

Furthermore, the myocardial infarction detection unit 27″ includes a myocardial infarct detector 45 adapted to evaluate the obtained intracardiac electrograms to detect changes being consistent with a myocardial infarction and to determine a location of the myocardial infarction using the evaluation. In one embodiment, the myocardial infarct detector is adapted to determine a ST segment elevation of each obtained intracardiac electrogram and compare them with a reference template stored in a template memory 46 to detect an occurrence of a myocardial infarction and a location of the myocardial infarction from the result of the comparison. Alternatively, the reference impedance template may be stored in the storage unit 25. Moreover, the reference template can be obtained and created before the parameter monitoring session is initiated, e.g. the impedance measurement session, and updated periodically.

In this embodiment, it is determined whether the ST segment elevation is above a predetermined ST segment threshold and in such a case; it is determined to be consistent with the occurrence of a myocardial infarction. The intracardiac electrogram having the largest ST segment elevation of the ST segments being above the predetermined ST segment threshold is determined to indicate the location of the myocardial infarction. That is, the electrode and/or electrode combination providing the intracardiac electrogram curve having the largest ST segment elevation during a cardiac cycle is determined to be the electrode and/or electrode combination being closest to the detected myocardial infarction and, hence, the location of the myocardial can be determined. The myocardial infarct detector 45 is adapted to send an instruction or message to the controller 24 informing the controller 24 that a myocardial infarction has been detected and the location of the myocardial infarction, i.e. as defined by the electrode and/or electrode combination being determined to be closest to the detected myocardial infarction. In one embodiment, the amplitude of a cardiac signal is measured during a short interval after the detection of R-wave. For example, the interval is about 40-150 ms after the R-wave detection. Measured amplitude is compared with a predetermined reference amplitude value and when the measured amplitude exceeds the reference value, a myocardial infarction is indicated.

Moreover, the myocardial infarct detector may be adapted to, after an initiation of a therapy session, monitor intracardiac electrograms obtained by at least an electrode and/or an electrode combination indicating the location of the myocardial infarction to determine whether obtained intracardiac electrograms indicate that the therapy session should be terminated and/or the treatment parameters should be adjusted. For example, a higher light intensity can be used during an initial therapy period and the light intensity can be reduced during a second period after the initial period. Alternatively, a constant light intensity but an adjusted intermittence can be utilized, e.g. the periods of light delivery can be adjusted or shorter intervals between the periods of light delivery are used.

The myocardial infarct detector may be adapted to determine ST segments for successive cardiac cycles after the initiation of the therapy session and to determine that therapy session should be terminated if a predetermined number of the obtained ST segment elevations are found to be below a predetermined ST segment elevation threshold. Alternatively, the therapy parameters can be adjusted, for example, shorter intervals between the periods of light delivery can be used if a predetermined number of the impedance value ratios are found to be above a predetermined impedance value ratio threshold.

According to a further embodiment of the present invention, the myocardial infarction detection means 27 comprises circuitry for detecting the occurrence and location of a myocardial infarction using both impedances and intracardiac electrogram. In this case, the occurrence and location of a myocardial infarction can be detected by using impedances and the healing process can be monitored by means of intracardiac electrograms, for example, by evaluating the ST elevation.

With reference to FIG. 4, an embodiment of a medical lead in accordance with the present invention will be described. The medical lead 50 comprises a number of tines 51 for fixating the lead 50 at the cardiac tissue.

An annular tip electrode 52 is arranged at the tip of the lead and will, after the implantation, abut against the cardiac tissue. A light emitting diode 54 is arranged at the centre of the tip portion of the lead. Further, an array of ring electrodes 55a-55c are arranged along an outer periphery 56 of the medical lead. An array of light emitting diodes 57a-57d is arranged along the outer periphery 56.

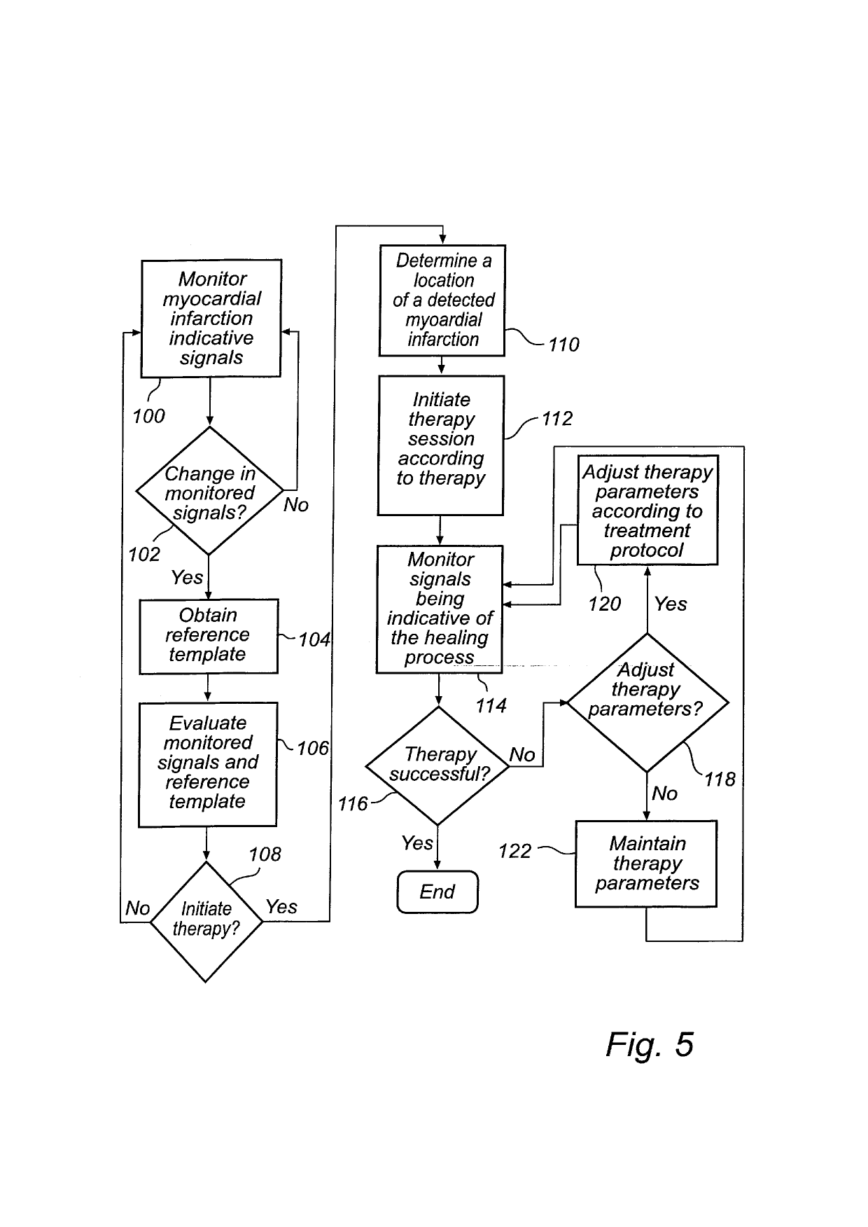

Turning now to FIG. 5, a high-level flow chart of an embodiment of the method for treating a myocardial infarction of a heart of a patient with therapeutic light using an implantable medical device according to the present invention is shown. At step 100, signals indicative of a myocardial infarction is monitored, for example, impedances of cardiac tissue or electrical activity for determining intracardiac electrograms as discussed above. This monitoring, i.e. the measuring or sensing sessions can be initiated at periodic intervals or can be performed continuously. At step 102, a determination is constantly or at regular intervals made in the myocardial infarct detector 35, 45 as to whether there has been any change in the signal being monitored of sufficient magnitude to indicate the possibility of an occurrence of a myocardial infarction. If no change, or if the magnitude of the change is too small, the algorithm returns to step 100. According to an embodiment, the algorithm waits for a predetermined period of time before it returns to step 100.

On the other hand, if a change that indicates the occurrence of a myocardial infarction is detected, the algorithm proceeds to step 104 where a reference template is obtained. The reference template may be a predetermined template stored in the template memory 36, 46, in the memory of the implantable medical device 20, or a template obtained and created by using measurements performed during a period when no myocardial indicative change in the monitored signals is detected. This created template may be updated periodically. Then, at step 106, the obtained data, e.g. the morphology of the impedance curves, a maximum impedance time derivative for the different impedance curves, or a ST elevation of the different intracardiac electrograms, are compared with the reference template. At step 108, it is checked whether the comparison indicates a deviation such that an occurrence of a myocardial infarction can be established and, thus, whether a delivery of therapy is justified. If the comparison indicates that the deviation is not sufficient to justify an initiation of a therapy, the algorithm returns to step 100.

If the deviation indicates that therapy should be initiated, the algorithm proceeds to step 110, where a location of the established myocardial detection is determined by using the obtained data, for example, the impedance curves or the intracardiac electrograms as described above. For example, the ST elevation being the largest or the minimum difference between the maximum impedance value and the minimum impedance value indicate which electrode and/or electrode combination that is closest to the detected myocardial infarction. Then, at step 112, a therapy session is initiated in accordance with a therapy protocol, which may include predetermined or adjustable treatment parameters such as emitting intervals of the therapeutic light, intensity of the emitted therapeutic light, wavelength of the emitted light, or intermittence of the emitted therapeutic light. The therapy protocol may be stored in the storage unit 25 of the implantable medical device 20 or in the memory of the myocardial detection means 27′, 27″.

At step 114, signals being indicative of the healing process are continuously monitored after the initiation of the therapy session. As described above, impedance signals and/or intracardiac electrograms may be used for this determination. At step 116, it is determined whether the therapy should be terminated based on the therapy protocol. If yes, the therapy is ended. On the other hand if no, the algorithm proceeds to step 118, where it is checked whether the therapy parameters should be adjusted. For example, shorter intervals between the periods of light delivery can be used if a predetermined number of the impedance value ratios, i.e. for a number of successive cardiac cycles, are found to be within a predetermined impedance value ratio interval or if the ST elevation, i.e. for a number of successive cardiac cycles, is found to be within a predetermined ST elevation value interval. If yes, the algorithm proceeds to step 120, where the therapy parameters are adjusted in accordance with the therapy protocol. Then, the algorithm returns to step 114, where the therapy is continued with the new adjusted parameters.

Alternatively, the algorithm may proceed to step 112, where a new therapy session is initiated with the new adjusted parameters. On the other hand, if it is determined that the therapy parameters should not be adjusted at step 118, the algorithm proceeds to step 122 where the therapy parameters are maintained. Thereafter, the algorithm returns to step 114, where the therapy is continued with the maintained therapy parameters. Alternatively, the algorithm may proceed to step 112, where a new therapy session is initiated with the maintained parameters.

The present invention applies to implantable medical devices such as implantable pacemakers including bi-ventricular pacemakers, pacemakers capable of delivering pacing to the atrium, the ventricle, or both the atrium and the ventricle (i.e. left ventricle and/or right ventricle), as well as devices, which are capable of delivering one or more cardioversion or defibrillation shocks.

In a further embodiment of the present invention, the control circuit 24 is adapted to, at detection of an occurrence of a myocardial infarction, send a notification to a medical care institution, e.g. a hospital or a care centre, via a communication unit of the medical device 10, 20, 30 and at least one external radio communication network such as wireless LAN (“Local Area Network”), GSM (“Global System for Mobile communications”), or UMTS (“Universal Mobile Telecommunications System”). For a given communication method, a multitude of standard and/or proprietary communication protocols may be used. For example, and without limitation, wireless (e.g. radio frequency pulse coding, spread spectrum frequency hopping, time-hopping, etc.) and other communication protocols (e.g. SMTP, FTP, TCP/IP) may be used. Other proprietary methods and protocols may also be used. The notification may include at least the patient identity, the occurrence of a myocardial infarction and/or the location of the detected infarct within the heart. The communication unit may be adapted to communicate with an extracorporeal communication device, e.g. mobile phone, a pager or a PDA (“Personal Digital Assistant”), which is adapted to receive the notification and to transmit it via said communication network further to the medical care institution. Alternatively, the communication unit may be adapted to communicate with a home monitoring unit located in the home of the patient. The home monitoring unit is adapted to communicate with the care institution via a telephone link. Furthermore, the notification may include a geographical location of the patient, for example, by means of a GPS (“Global Positioning System”) unit arranged in the communication device. Thereby, it is possible for the care institution to obtain an early notification of the infarct of a patient and, additionally, the position of the patient and hence the patient can be given care at an early stage of an infarction.

In a further embodiment of the present invention, an extracorporeal therapy unit may be connected to a medical lead according to the present invention for supplying, for example, power to the light emitting means or, in case of light conducting optical fibres in the medical lead for supplying therapeutic light. Furthermore, an extracorporeal therapy unit comprising a lead in form of a guide wire including light emitting means in accordance with the present invention may be used to treat the detected infarct since the medical personnel controlling the therapy unit may be provided with the location of the detected infarct via the implanted medical device.

Although modifications and changes may be suggested by those skilled in the art, it is the intention of the inventors to embody within the patent warranted heron all changes and modifications as reasonably and properly come within the scope of their contribution to the art.

We claim as our Invention:

1. An implantable medical device comprising:
a pulse generator that emits cardiac stimulating pacing pulses;
a medical lead connected to said pulse generator that delivers said pulses in vivo to cardiac tissue of the heart of a patient;
a myocardial infarction detection unit that detects a myocardial infarction and identifies a location of said myocardial infarction;
a therapy circuit connected to a plurality of light emitting units carried by said medical lead, each of said light emitting units being operated by said therapy circuit to emit therapeutic light; and
a control circuit connected to said myocardial infarction detection unit and to said therapy circuit, said control circuit being configured to initiate a therapy session via said therapy circuit, in which one or more of said plurality of light emitting is/are selectively activated to emit said therapeutic light toward the detected location of the myocardial infarction upon detection of an occurrence of the myocardial infarction.

2. The implantable medical device according to claim 1, wherein said therapy circuit is configured to activate said light emitting units to emit said therapeutic light according to a treatment protocol.

3. The implantable medical device according to claim 2, wherein said treatment protocol includes treatment parameters comprising: emitting intervals of said therapeutic light, intensity of said emitted therapeutic light, wavelength of said emitted light, and intermittence of said emitted therapeutic light.

4. The implantable medical device according to claim 1, wherein each of said plurality of light emitting units comprises at least one light emitting diode.

5. The implantable medical device according to claim 1, wherein said light emitting units are arranged in an array arranged along an outer surface of a lead body of said medical lead.

6. The implantable medical device according to claim 1, wherein each of said plurality of light emitting units comprises at least one optical fibre fiber that conducts light emitted from at least one light source said implantable medical device, and wherein said therapy circuit selectively activates said at least one light source and/or at least one optical fiber to cause light conducted in one or more optical fiber to emanate from said one or more optical fiber toward said detected location.

7. The implantable medical device according to claim 6, wherein the at least one light source is a laser source.

8. The implantable medical device according to claim 1, wherein said medical lead comprises a plurality of electrodes, and wherein said myocardial infarction detection unit comprises:
an impedance measuring circuit connected to said electrodes and configured to:
apply excitation current pulses between respective electrode pairs including at least a first and at least a second electrode; and
measure the impedance in the tissues between said at least first and said at least second electrode of said electrode pairs to the excitation current pulses; and
a myocardial infarct detector that evaluates said measured impedances by detecting changes in said impedances associated with the myocardial infarction and to determine the location of said myocardial infarction using said evaluation.

9. The implantable medical device according to claim 8, wherein said myocardial infarct detector configured to compare measured impedances with a stored reference impedance template to detect the occurrence of the myocardial infarction and the location of said myocardial infarction from a result of the comparison.

10. The implantable medical device according to claim 9, wherein said myocardial infarct detector configured to:
determine impedance value ratios for a cardiac cycle by determining a maximum impedance and a minimum impedance, respectively, measured by the impedance measuring circuit during a cardiac cycle;
determine an impedance value ratio being below a predetermined impedance value ratio threshold to be consistent with a myocardial infarction; and
determine the impedance value ratio being smallest of the impedance value ratios being below said predetermined impedance value ratio threshold to indicate the location of the myocardial infarction.

11. The implantable medical device according to claim 9, wherein said myocardial infarct detector is configured to:
calculate a respective maximum time derivative of the measured impedance curves;
determine a maximum impedance time derivative being below a predetermined impedance time derivative threshold to be consistent with a myocardial infarction; and
determine the maximum impedance time derivative being lowest of the maximum impedance time derivatives being below said predetermined impedance time derivative threshold to indicate the location of the myocardial infarction.

12. The implantable medical device according to claim 1, wherein said medical lead comprises a plurality of electrodes, and wherein said myocardial infarction detection unit comprises:
an intracardiac electrogram measuring circuit connected to said electrodes that measures intracardiac electrograms using respective pairs of said electrodes; and
a myocardial infarct detector that evaluates said intracardiac electrograms to detect changes consistent with a myocardial infarction and to determine the location of said myocardial infarction using said evaluation.

13. The implantable medical device according to claim 12, wherein said myocardial infarct detector is configured to:
determine a ST segment elevation being above a predetermined ST segment threshold as being consistent with the occurrence of a myocardial infarction; and
determine the intracardiac electrogram having the largest ST segment elevation of the ST segments being above a predetermined ST segment threshold as indicating the location of said myocardial infarction.

14. The implantable medical device according to claim 8, wherein said myocardial infarct detector is, after an initiation of a therapy session, configured to:
monitor impedances obtained by at least an electrode pair indicating the location of said myocardial infarction to determine whether said impedances indicate that said therapy session should be terminated and/or said treatment parameters should be adjusted.

15. The implantable medical device according to claim 14, wherein said myocardial infarct detector is adapted configured to:
determine impedance value ratios for successive cardiac cycles; and
determine that said therapy session should be terminated if a predetermined number of said impedance value ratios are found to be above said impedance value ratio threshold.

16. The implantable medical device according to claim 14, wherein said myocardial infarct detector is configured to:
calculate maximum time derivatives of the measured impedance curves for successive cardiac cycles; and
determine that said therapy session should be terminated if a predetermined number of said maximum impedance time derivatives are found to be above a predetermined impedance time derivative threshold.

17. The implantable medical device according to claim 12, wherein said myocardial infarct detector is configured to:
monitor intracardiac electrograms obtained by at least an electrode pair indicating the location of said myocardial infarction to determine whether said intracardiac electrograms indicate that said therapy session should be terminated and/or said treatment parameters should be adjusted.

18. The implantable medical device according to claim 17, wherein said myocardial infarct detector is configured to:
determine ST segments for successive cardiac cycles; and
determine that said therapy session should be terminated if a predetermined number of said ST segment elevations are found to be below a predetermined ST segment elevation threshold.

19. The implantable medical device according to claim 1, wherein each of said light emitting units emits coherent and monochromatic light.

20. The implantable medical device according to claim 1, wherein each of said light emitting unit emits light having a wavelength in the range of 600 nm-1000 nm.

21. The implantable medical device according to claim 1 comprising a communication unit, and wherein said control circuit is configured to, upon detection of an occurrence of a myocardial infarction, send a notification to a medical care institution via said communication unit of and at least one external communication network, said notification including at least an identity of the patient and information related to a detected myocardial infarction.

22. The implantable medical device according to claim 21, wherein said communication unit 30) is configured to communicate with an extracorporeal communication device, said communication device being configured to receive said notification and to transmit said notification via said communication network to said medical care institution.

23. The implantable medical device according to claim 22, wherein said extracorporeal communication device is a mobile phone, a pager or a PDA (“Personal Digital Assistant”).

24. The implantable medical device according to claim 21, wherein said communication unit of said medical device is configured to communicate with an extracorporeal home monitoring unit connected to said at least one communication network, said home monitoring unit being adapted to receive said notification and to transmit said notification via said communication network to said medical care institution.

25. The implantable medical device according to claim 1, further comprising a notifying device configured to, upon detection of an occurrence of myocardial infarction, notify said patient that the myocardial infarct has been detected and/or that therapy has been initiated.

26. The implantable medical device according to claim 25, wherein said notifying device is a vibration unit.

27. A method for treating cardiac tissue of a heart of a patient with therapeutic light using an implantable medical device including a pulse generator that emits cardiac stimulating pacing pulses and connectable to at least one medical lead for delivering said pulses in vivo to cardiac tissue of a heart of a patient, comprising the steps of:
detecting in vivo a myocardial infarction and identifying a location of said myocardial infarction; and
automatically initiating an in vivo therapy session by selectively activating one or more of a plurality of light emitting units carried said at least one medical lead to emit therapeutic light detected location of the myocardial infarction upon detection of an occurrence of the myocardial infarction.

28. The method according to claim 27, further comprising the step of:
selectively activating said light emitting units to emit said therapeutic light according to a treatment protocol.

29. The method according to claim 28, wherein said treatment protocol includes treatment parameters comprising: emitting intervals of said therapeutic light, intensity of said emitted therapeutic light, wavelength of said emitted light, and intermittence of said emitted therapeutic light.

30. The method according to claim 27 comprising forming each of said plurality of light emitting units as at least one light emitting diode.

31. The method according to claim 27, comprising arranging said light emitting units in an array arranged along an outer surface of a lead body of said medical lead.

32. The method according to claim 27, further comprising the step of:
emitting said therapeutic light via at least one optical fiber that conducts light from at least one light source in said implantable medical device to cause said conducted therapeutic light to emanate from said at least one optical fiber toward the detected location of the myocardial infarction upon detection of the occurrence of the myocardial infarction.

33. The method according to claim 32, comprising employing a laser source as said at least one light source.

34. The method according to claim 27, wherein the step of detecting a myocardial infarction and identifying a location of said myocardial infarction comprises the steps of:
applying excitation current pulses between respective electrode pair including at least a first and at least a second electrode;
measuring the impedance in the tissues between said at least first and said at least second electrode of said electrode pairs to the excitation current pulses;
evaluating said measured impedances by detecting changes in said impedances being consistent with a myocardial infarction; and
determining a location of said myocardial infarction using said evaluation.

35. The method according to claim 34, wherein the step of evaluating comprises the step of:
comparing measured impedances with a stored reference impedance template to detect an occurrence of a myocardial infarction and a location of said myocardial infarction from the result of the comparison.

36. The method according to claim 35, wherein the step of comparing comprises the steps of:
determining impedance value ratios for a cardiac cycle by determining a maximum impedance and a minimum impedance, respectively, measured by the impedance measuring circuit during a cardiac cycle;
determining an impedance value ratio being below a predetermined impedance value ratio threshold to be consistent with a myocardial infarction; and
determining the impedance value ratio being smallest of the impedance value ratios being below said predetermined impedance value ratio threshold to indicate the location of the myocardial infarction.

37. The method according to claim 35, wherein the step of comparing comprises the steps of:
calculating a respective maximum time derivative of the measured impedance curves;
determining a maximum impedance time derivative being below a predetermined impedance time derivative threshold to be consistent with a myocardial infarction; and
determining the maximum impedance time derivative being lowest of the maximum impedance time derivatives being below said predetermined impedance time derivative threshold to indicate the location of the myocardial infarction.

38. The method according to claim 27, wherein said medical lead comprises a plurality of electrodes, and further comprising the steps of:
measuring intracardiac electrograms using respective pairs of said electrodes; and
evaluating said intracardiac electrograms to detect changes being consistent with a myocardial infarction and to determine a location of said myocardial infarction using said evaluation.

39. The method according to claim 38, wherein the step of evaluating comprises the steps of:
determining a ST segment elevation being above a predetermined ST segment threshold as being consistent with the occurrence of a myocardial infarction; and
determining the intracardiac electrogram having the largest ST segment elevation of the ST segment elevations being above a predetermined ST segment threshold as indicating the location of said myocardial infarction.

40. The method according to claim 35, further comprising the step of:
monitoring impedances obtained by an electrode pair indicating the location of said myocardial infarction to determine whether said impedances indicate that said therapy session should be terminated and/or said treatment parameters should be adjusted.

41. The method according to claim 40, further comprising the steps of:
determining impedance value ratios for successive cardiac cycles; and
determining that said therapy session should be terminated if a predetermined number of said impedance value ratios are found to be above said impedance value ratio threshold.

42. The method according to claim 40, further comprising the steps of:
calculating maximum time derivatives of the measured impedance curves for successive cardiac cycles; and
determining that said therapy session should be terminated if a predetermined number of said maximum impedance time derivatives is found to be above a predetermined impedance time derivative threshold.

43. The method according to claim 35, further comprising the step of:
monitoring intracardiac electrograms obtained by an electrode pair indicating the location of said myocardial infarction to determine whether said intracardiac electrograms indicate that said therapy session should be terminated and/or said treatment parameters should be adjusted.

44. The method according to claim 43, further comprising the steps of:
determining ST segments for successive cardiac cycles; and
determining that said therapy session should be terminated if a predetermined number of said ST segment elevations are found to be below a predetermined ST segment threshold.

45. The method according to claim 27 comprising, from said light emitting unit emitting coherent and monochromatic light.

46. The method according to claim 27 comprising, from said light emitting unit emitting light having a wavelength in a range of 600 nm-1000 nm.

47. The method according to claim 27, further comprising the step of, upon detection of an occurrence of the myocardial infarction, sending a notification to a medical care institution via a communication unit of said medical device and at least one external communication network, and including in said notification including at least an identity of the patient and information related to a detected myocardial infarction.

48. The method according to claim 47, wherein the step of sending a notification comprises the steps of:
communicating with an extracorporeal communication device and, at said communication device receiving said notification and transmitting said notification via said communication network to said medical care institution.

49. The method according to claim 48 comprising employing, as said extracorporeal communication device, a mobile phone, a pager or a PDA (“Personal Digital Assistant”).

50. The method according to claim 48, wherein the step of sending a notification comprises the step steps of:
communicating with an extracorporeal home monitoring unit connected to said at least one communication network and, at said home monitoring unit, receiving said notification and transmitting said notification via said communication network to said medical care institution.

51. The method according to claim 27, further comprising the step of, upon detection of an occurrence of the myocardial infarction, automatically notifying said patient that the myocardial infarct has been detected and/or that therapy has been initiated.

52. The method according to claim 51, comprising notifying the patient via a vibration unit.

53-59. (canceled)

60. A computer-readable medium encoded with programming instructions, said medium being loadable into a control unit of an implantable medical device comprising a pulse generator that emits cardiac stimulation pulses and at least one medical lead connected to the pulse generator for delivering the stimulating pulses in vivo to cardiac tissue of a heart of a patient, and a plurality of light emitting units carried by the at least one medical lead, said programming instructions causing said control unit to:
detect in vivo a myocardial infarction and to identify a location of the myocardial infarction; and
initiate an in vivo therapy session by selectively activating one or more of said light emitting units to emit therapeutic light toward the detected location of the myocardial infarction upon detection of an occurrence of the myocardial infarction.
